SEQUENCE LISTING (4 sequences)

SEQ ID NO: 1 - protein, 287 aa, Artificial
  based on fragments of influenza viral sequences
RIYQILAIYITIGKCPKYSLLTEVETYGPGPGTSADQQSLYGPGPGEMNAPNYHYLASCMGLIYDTVNRTHQYGTEKLTITYAFDERRNKYDTVHDRTPYLTEVETYVLGPGPGSVKNGTYDYGTIKDRSPYNADTLCIGYGPGPGGMVDGWYGYCTELKLSDYGPGPGVSDGGPNLYEICPKLAEYKSCINRCFYQVSRPMFLYGAKEVALSYGPGPGELDAPNYHYDLLENLQAYASQGTKRSYNTDLEALMENMDKAVKLYLSTASSWSYGPGPGGMIDGWYGY

SEQ ID NO: 2 - protein, 394 aa, Artificial
  based on fragments of influenza viral sequences
TRGVQIASNENVETMDSNTLELRTYVLSIIPSGPLKAEIAQRLESVYRYGFVANFSMELPSFGVSGCRTFFLTQGALLNDKHIDPFKLLQNSQVVSLMRPYEELREQLSSVSSFERFQDILMRMSKMQLGSSSEMMGMFNMLSTVLGVSIGDKITFEATGNLVVPRYRSKFLLMDALKLSIEDPSCMGLIYNRMGTVTTEAAFGLVCFEQITFMQALQLLLEVENQTYVNISNTNFAAGQSVVSVKLVPRYAFAMERNAGSGIIISEVHIYYLEKANKIKSEKTHIHIFGPGPGMANLILQIGNIISIWISHSIEDLIFLARSALILRGSVAHKSCLPTRQMVHAMRTIGTHPSSSASVVSVKLAGNSSLCPVSTRIYQILAIYSTVASSLVLV

SEQ ID NO: 3 - DNA, 867 bp, Artificial
  reverse-transcribed from artificial protein sequence
cggatctaccagattctggccatctacatcaccatcggcaagtgccccaagtactctctgctgaccgaagtggaaacatatggccccggacccggcaccagtgccgatcagcagtctctgtacggacccggacccggggagatgaacgcccccaactatcactatctggccagctgcatgggactgatctacgacaccgtgaaccggacccaccagtacggcaccgagaagctgaccatcacatacgccttcgacgagcggcggaacaaatacgataccgtgcacgaccggaccccctatctgactgaagtggaaacttacgtgctgggacccggacccggcagcgtgaagaatggcacatacgactacggcaccatcaaggacagaagcccctacaacgccgacacactgtgcatcggctatgggcccggacccgggggaatggtggatgggtggtacggctactgcaccgagctgaagctgagcgactatggacccggacccggcgtgtcagatggcggcccaaatctgtacgaaatctgccccaagctggccgagtacaagagctgcatcaaccggtgcttctaccaagtgtcccggcccatgtttctgtacggcgccaaagaagtggctctgagctacggacccggacccggcgaactggatgccccaaactaccactacgatctgctggaaaatctgcaagcctacgccagccaaggcaccaagcggagctacaacaccgatctggaagctctgatggaaaacatggacaaggccgtgaagctgtatctgagcaccgccagcagctggtcttacggacccggacccggcggaatgatcgacgggtggtatggatactgatga

SEQ ID NO: 4 - DNA, 1188 bp, Artificial
  reverse-transcribed from artificial protein sequence
acaagaggcgtgcagatcgccagcaacgagaacgtggaaaccatggacagcaacacactggaactgcggacatacgtgctgagcatcatccccagcggccctctgaaggccgagatcgcccagagactggaaagcgtgtacagatacggcttcgtggccaacttcagcatggaactgcccagcttcggcgtgtccggctgcagaacattctttctgacacaaggcgctctgctgaacgacaagcacatcgaccccttcaagctgctgcagaatagccaagtggtgtctctgatgcggccctacgaggaactgcgcgagcagctgtccagcgtgtccagcttcgagcggttccaagacattctgatgcgcatgagcaagatgcagctgggctccagcagcgagatgatgggcatgttcaatatgctgagcaccgtgctgggcgtgtccatcggcgacaagatcacattcgaggccaccggcaatctggtggtgccccggtacagatccaagtttctgctgatggacgctctgaagctgtccatcgaggaccctagctgcatgggactgatctacaaccggatgggcaccgtgaccaccgaggccgcttttggactcgtgtgcttcgagcagatcactttcatgcaagctctgcagctgctgctggaagtggaaaaccagacttacgtgaacatcagcaacaccaacttcgccgctggccagagcgtggtgtccgtgaagctggtgcccagatacgcctttgccatggaacggaatgccggcagcggcatcatcatcagcgaagtgcacatctactatctggaaaaggccaacaagatcaagagcgagaaaacccacatccacatctttggccccggacccggcatggccaatctgattctgcagatcggcaacatcatctccatctggatcagccactctatcgaggatctgatctttctggctcggagcgcactgattctgagaggcagcgtggcccacaagagctgtctgcccaccagacagatggtgcacgccatgagaaccatcggcacccaccctagctctagcgcctccgtggtgtctgtgaaactggccggcaatagctctctgtgccccgtgtccacccggatctaccagattctggccatctacagcaccgtggcctcttctctggtgctcgtgtgatga

(19) United States
(12) Patent Application Publication
(10) Pub. No.: US 2022/0023412 A1
(43) Pub. Date: Jan. 27, 2022

(54) Compositions Useful in Both Homologous And Heterologous Vaccine Regimens

(71) Applicants: University of Rhode Island Board of Trustees, Kingston, RI (US); EpiVax, Inc., Providence, RI (US)

(72) Inventors: Anne De Groot, Providence, RI (US); Leonard J. Moise, Providence, RI (US); William Martin, Providence, RI (US)

(21) Appl. No.: 17/355,543

(22) Filed: Jun. 23, 2021

Related U.S. Application Data
(60) Provisional application No. 63/042,559, filed on Jun. 23, 2020.

(51) Int. Cl. A61K 39/145; A61P 31/16

(52) U.S. Cl. CPC A61K 39/145; A61K 2039/5252; A61P 31/16

(57) ABSTRACT

The invention provides a swine influenza vaccine composition as part of a vaccine package where the composition produces one or more T cell epitopes that are highly conserved in circulating influenza strains including those of H1N1 and others. The invention addresses variability amongst the influenza viruses from year to year, and therefore, provides great economic relief to pork farmers. The invention further provides a preferred, heterologous vaccine regimen embodiment where a DNA vaccine, such as the composition disclosed herein, is used in combination with a more conventional vaccine, such as one based on part or whole of an inactivated virus.

### CROSS REFERENCE TO RELATED APPLICATION

This application claims priority to and the benefit of co-pending U.S. provisional patent application Ser. No. 63/042,559, filed Jun. 23, 2020, which application is incorporated herein by reference in its entirety.

### REFERENCE TO SEQUENCE LISTING

The instant application contains a Sequence Listing which has been submitted electronically in ASCII format and is hereby incorporated by reference in its entirety. Said ASCII text file, created Jun. 22, 2021, is named “URI2002-Seq_ST25.txt” and is 9 KB bytes in size.

### FIELD OF THE INVENTION

The invention relates to swine influenza vaccine compositions, treatment regimens and related methods.

### BACKGROUND OF THE INVENTION

When pigs contract a respiratory disease, both the welfare of the pigs and their economic value suffer, along with pig farmers. These diseases are often caused by viruses, such as Influenza A virus (IAV), which is an enveloped virus of the Orthomyxoviridae family, with segmented negative-sense single-strand RNA. The virus in pigs, often referred to as swine influenza, swine flu, swine IAV or IAV-S, is highly contagious in pigs and transmitted through direct contact and (nasal) mucus aerosols from pig to pig. Clinical indications that a pig may have contracted the IAV include fever, loss of appetite, lethargy, paroxysmal coughing, sneezing, nasal discharge, and conjunctivitis. The prevalence of IAV as a reason for acute respiratory disease or endemic infections in pig herds is probably underestimated (de Jong, J C. et al. Vaccine (1999) 17: 1321-1328). Swine influenza impacts pig farmers' income because it brings weight loss, reduced weight gain and predisposition to other infections (Opriessnig T et al., Anim Health Res Rev Conf Res Work Anim Dis. (2011) 12: 133-148), as well as reproductive failures secondary to fever in breeding herds (Rajao D S et al., Curr Top Microbiol Immunol. (2014) 385: 307-326).

Recently, much more attention has been paid to IAV in pigs because of the pandemic IAV outbreak in 2009, which highlighted concern over the ability of avian, porcine and human influenza viruses to re-assort genes from other IAV subtypes (called “antigenic shift”) and create new strains, in particular, those with greater pathogenic virulence (Stincarelli M et al., Virus Res (2013) 175: 151-154). Human-to-swine “spillover” events also contribute to the genetic diversity of swine IAV (Nelson M I et al., Trends Microbiol. (2015) 23: 142-153). North American subtypes of IAV that commonly circulate in the pig population include H1N1, H1N2, and H3N2 (Anderson T K et al., Influenza Other Respir Viruses (2013) 7: 42-51); moreover, IAV is ubiquitous and widely transmitted worldwide in a wide array of strains.

Within a subtype, influenza strains can evolve to vary in the form of small amino acid or glycosylation differences in the viral surface glycoproteins of hemagglutinin (HA) and neuraminidase (NA). These mutations are positively selected for by immune pressure, as evident through studies of pandemic strains. These slight changes in important viral epitopes occur with great ease and frequency, and lead to antigenic changes in HA or NA known as “antigenic drift” (Kim, H. et al., Viral Immunol. 2018; 31: 174-183). As a result, vaccines targeting a particular influenza strain and those antigenically close ones often turn out to be compromised in efficacy when the next epidemic breaks out. Further complicating the situation is the zoonotic potential of IAV where a swine IAV can infect other species including human.

Accordingly, there remains an unmet need for cross-protective vaccines and related regimens that can offer heterosubtypic, or “universal” immunity against IAV for one or more species such as pig and/or human. It is imperative that we came up with new vaccines and methods of administration that provide improved breadth and strength against an ever-evolving viral target.

### SUMMARY OF THE INVENTION

In one aspect, the invention provides a swine influenza vaccine package that includes: (a) a first vaccine having nucleic acid molecules, e.g., one or more DNA molecules, that lead to the production, in vitro or in vivo, of polypeptides sharing at least 80%, 85%, 90%, 95% or 100% homology with at least one of SEQ ID NO:1 and SEQ ID NO:2; and (b) a second inactivated swine influenza vaccine for inoculation of the same subject. In a preferred feature, the first vaccine leads to the production of the entire polypeptides of SEQ ID NOS:1 and 2. In one embodiment, the first vaccine includes DNA sequences that share at least 80%, 85%, 90%, 95% or 100% homology with at least one of DNA sequences represented in SEQ ID NOS:3 and 4 or of a functional equivalent thereof. In one feature, the first vaccine includes DNA molecules with either naturally occurring deoxyribonucleotide residues or modified deoxyribonucleotide residues.

In an embodiment, the DNA sequences in the first vaccine that lead to the production of polypeptides sharing (i) at least 80-100% homology with SEQ ID NO:1 and (ii) at least 80-100% homology with SEQ ID NO:2, can be in separate DNA molecules or fused into one molecule. The first vaccine preferably is effective in eliciting a significant cell-mediated immunity response to a challenge from one or more swine influenza strains. The second vaccine preferably is effective in eliciting a significant humoral immunity response to the swine influenza challenge and can be, e.g., a whole inactivated virus vaccine, a split virus vaccine, a subunit vaccine, a live attenuated vaccine, and a virosome vaccine. The vaccine package is designed to be administered as a prime-boost regimen and for eliciting immunity against one or more influenza viral strains, e.g., those selected from H1N1, H1N2, and H3N2. In an embodiment, the vaccine is for use in a pig. In a feature, the vaccine of the invention is formulated and packaged for intradermal administration. In another feature, the vaccine further includes an adjuvant.

In a preferred embodiment, a swine influenza vaccine package for inoculating a pig is provided, where said package includes a first vaccine and a second vaccine: said first vaccine includes a first DNA molecule where its sequence consists of SEQ ID NO:3, and a second DNA molecule where its sequence consists of SEQ ID NO:4; and said second vaccine comprises a whole inactivated virus vaccine against swine influenza.

In a further aspect, the invention provides a prime-boost regimen or method of inoculating a pig against swine influenza virus through the steps of administering to a pig facing potential exposure to one or more influenza strains an immunogenically effective amount of a first nucleic acid (e.g., DNA or RNA) vaccine composition of the present invention and an immunogenically effective amount of a second swine influenza vaccine separated by an interdose interval, e.g., at least 24 hours or one week. In an embodiment, the first nucleic acid vaccine is administered first, and the second swine influenza vaccine is administered at a time that is at least one, two or three weeks later. In a feature, at least one of the vaccines is administered intradermally. In another embodiment, the second swine influenza vaccine is administered first and before the first nucleic acid vaccine is administered. In a heterologous prime-boost embodiment, the second vaccine comprises an inactivated swine influenza vaccine. In a homologous prime-boost embodiment, the second vaccine comprises the same nucleic acid vaccine as the first.

In yet another aspect of the invention, a method of inoculating a pig against a swine influenza virus challenge is provided, where the method includes:
a. administering to a pig facing potential exposure to one or more swine influenza strains an immunogenically effective amount of a first vaccine to effect a cell-mediated immunity response to a challenge from one or more swine influenza strains; and
b. administering to the same pig an immunogenically effective amount of a second and different vaccine separated by an interdose interval, e.g., at least a day, wherein said second vaccine effects a humoral immunity response to the viral challenge.

In various embodiments, the first vaccine may be a nucleic acid, e.g., DNA, vaccine that produces in vivo at least one T cell epitope conserved among multiple strains of swine IAV, and the second vaccine may be a viral-protein-based vaccine, e.g., an inactivated vaccine based on a swine influenza protein or peptide. In an embodiment, said first vaccine is administered first, followed by said second vaccine after an interdose interval.

The foregoing and other objects, aspects, features, and advantages of the invention will become more apparent from the following description and from the claims.

### BRIEF DESCRIPTION OF THE DRAWINGS

The objects and features of the invention can be better understood with reference to the drawings described below, and the claims. The drawings are not necessarily to scale, emphasis instead generally being placed upon illustrating the principles of the invention.

FIG. 1 list representative circulating swine influenza strains of which sequences were searched and accessed in making an exemplary vaccine embodiment of the invention.

FIG. 2 shows analyses by immunoinformatics tools of 28 candidate T-cell epitopes in class I SLA alleles conserved across IAV strains investigated according to aspects of the invention.

FIG. 3 shows analyses by immunoinformatics tools of 20 candidate T-cell epitopes in class II SLA alleles conserved across IAV strains investigated according to the invention.

FIG. 4 illustrates that the conservation between an epitope-vaccine-driven vaccine and circulating IAV strains remained steady over the years between 2013 and 2017.

FIG. 5A provides a flowchart of steps taken to arrive at a vaccine embodiment of the invention. FIG. 5B shows epitope sequence embodiments resulting from the process depicted in FIG. 5A. FIG. 5C shows examples of DNA sequence embodiments derived from the embodiments shown in FIG. 5B.

FIG. 6 shows treatment groups' setup for collecting data regarding a vaccine embodiment of the invention.

FIG. 7 lists the 72 swine IAV pH1N1 2017 isolates from which a particular stain was selected as the basis for making a vaccine embodiment of the invention.

FIGS. 8A and 8B summarize the SLA class I (28) and class II (20) epitopes (SEQ ID NOS. 3-30 and NOS. 31-50, respectively) identified through an immunoinformatics tool in the process of making a vaccine embodiment of the invention. [^{a}The numerator is the count of JanusMatrix matches found in the search database (swine IAV strains). With respect to a given EpiMatrix Hit (a 9-mer contained within the input sequence which is predicted to bind to a specific allele), a JanusMatrix match is a 9-mer derived from the search database which is predicted to bind to the same allele as the EpiMatrix Hit and shares TCR facing contacts with the EpiMatrix Hit. The denominator is the number of EpiMatrix Hits per peptide across the 72 H1N1 IAV strain dataset shown in FIG. 7. ^{b}Janus Homology Score represents the average depth of coverage in the search database for each EpiMatrix hit in the input sequence. It considers all constituent 9-mers in any given peptide, including flanks.]

FIG. 9 shows a graphic summary of NP ELISA antibody response data for testing the vaccine embodiments of the invention according to setup in FIG. 6.

FIG. 10 is a graphic summary of data on post-vaccination CMI response from vaccine embodiments of the invention tested according to experiments outlined in FIG. 6, and shows the group mean numbers of IFNγ producing cells per million PBMC±SEM in the treatment stimulated by using the challenge virus or peptides at day post challenge (dpc)-1.

FIG. 11 is a graphic illustration of data on post-challenge body temperature from pigs under the test outlined according to FIG. 6, which shows their mean rectal temperatures±SEM in the different treatment groups at certain days post challenge with pH1N1 strains.

FIG. 12 is a graphic illustration of data on post-challenge nasal shedding from pigs under the test outlined according to FIG. 6, which shows RT-PCR data on IAV detected in nasal swabs at different days post IAV challenge.

FIGS. 13A and 13B are graphic illustrations of data on post-challenge macroscopic lung lesions (FIG. 13A) and IAV antigen data (FIG. 13B) from pigs under the test outlined according to FIG. 6.

FIG. 14 are photographic depicts of sample pig lungs at necropsy that were examined for macroscopic lesions under the test outlined according to FIG. 6.

### DETAILED DESCRIPTION OF THE INVENTION

### I. Definition

Unless otherwise noted, technical terms are used according to conventional usage. Definitions of common terms in molecular biology may be found, for example, in J. Krebs et al. (eds.), Lewin's Genes XII, published by Jones and Bartlett Learning, 2017 (ISBN 9781284104493); Robert A. Meyers (ed.), Molecular Biology and Biotechnology: a Comprehensive Desk Reference, published by Anmol Publications Pvt. Ltd, 2011 (ISBN 9788126531783); and other similar technical references.

As used in the specification and claims, the singular form “a”, “an”, or “the” includes plural references unless the context clearly dictates otherwise. For example, the term “a cell” includes a plurality of cells including mixtures thereof. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent,” or “except for [a particular feature or element],” or “wherein [a particular feature or element] is not present (included, etc.) . . . ”.

When a dimensional measurement is given for a part herein, the value is, unless explicitly stated or clear from the context, meant to describe an average for a necessary portion of the part, i.e., an average for the portion of the part that is needed for the stated purpose. Any accessory or excessive portion is not meant to be included in the calculation of the value.

As used herein, the recitation of a numerical range for a variable is intended to convey that the invention may be practiced with the variable equal to any of the values within that range. Thus, for a variable that is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values >0 and <2 if the variable is inherently continuous.

As used herein, “about” means within plus or minus 10%. For example, “about 1” means “0.9 to 1.1”, “about 2%” means “1.8% to 2.2%”, “about 2% to 3%” means “1.8% to 3.3%”, and “about 3% to about 4%” means “2.7% to 4.4%.”

As used herein, the term “adjuvant” refers to a substance or a combination of substances that non-specifically enhances the effectiveness of a vaccine. Typically, an adjuvant is formulated as part of a vaccine to enhance the vaccine's ability to induce protection in the body against infection by a pathogen, e.g., a virus or bacterium. Adjuvants are considered to enhance the immune response to an antigen in the vaccine in at least one of the following ways: prolong the presence of antigen in the body (blood and/or tissue); help antigen-presenting cells absorb antigen; activate macrophages and lymphocytes; and support the production of cytokines. Adjuvants are also used in the production of antibodies from immunized animals. An “adjuvant effect” refers to enhancement in the immune response to a selected antigen in a host that receives the vaccine.

As used herein, the terms “administration,” “administering,” or the like, when used in the context of providing a pharmaceutical composition to a subject generally refers to providing to the subject one or more pharmaceutical compositions comprising the agent, e.g., a vaccine, in combination with an appropriate delivery vehicle by any means such that the administered compound achieves one or more of the intended biological effects for which the compound was administered. By way of non-limiting example, a composition may be administered parenteral, subcutaneous, intravenous, intracoronary, rectal, intramuscular, intra-peritoneal, transdermal, intradermal or buccal routes of delivery.

As used herein, the term “immunogenically effective amount” in relation to the treatment of IAV and/or associated diseases (e.g., for one or more species such as pig and/or human) refers to the amount of the composition suitable for establishing an immune response against the IAV and/or associated diseases. The term “immune response” refers to the concerted action of lymphocytes, antigen presenting cells, phagocytic cells, granulocytes, and/or soluble macromolecules produced by the above cells or the liver (including antibodies, cytokines, and complement) that results in selective damage to, destruction of, or elimination from the subject of the invading pathogens and/or cells or tissues infected with pathogens.

As used herein, “anti-IAV activity”, “anti-IAV polypeptides”, “anti-IAV compositions”, “anti-IAV DNA vaccines” and the like are intended to mean that the T-cell epitope compounds and compositions of the present disclosure (including polypeptides, concatemeric polypeptides, nucleic acids such as the DNA vaccines as disclosed herein, plasmids, vectors, pharmaceutical compositions, vaccines, and other compositions of the instant disclosure) have anti-IAV activity and thus are capable of suppressing, controlling, and/or killing an invading Influenza A virus. For example, anti-IAV activity means that the instantly-disclosed therapeutic T-cell epitope compounds and compositions are, in aspects: capable of stimulating, inducing, and/or expanding an immune response to IAV (e.g., a cellular and/or humoral immune response to IAV) and/or associated diseases in a subject; capable of stimulating, inducing, and/or expanding an IAV-specific IFNγ response (e.g., by lymphocytes such as PMBC, or effector CD4+ and/or CD8+ T-cells), and/or capable of inducing immunity against IAV. In aspects, a T-cell epitope compound or composition of the present disclosure having anti-IAV activity will reduce the disease symptoms resulting from IAV challenge by at least about 5% to about 50%, at least about 10% to about 60%, at least about 30% to about 70%, at least about 40% to about 80%, or at least about 50% to about 90% or greater, including any value or range therebetween. Anti-IAV activity can be determined by various experiments and assays as known to those of skill in the art, including methods such as by antibody titrations of sera, e.g., by ELISA and/or seroneutralization assay analysis and/or by vaccination challenge evaluation, including the experiments and assays as disclosed in the Examples herein.

As used herein, the term “polypeptide” refers to a linear organic polymer consisting of a number of amino-acid residues bonded together in a chain.

As used herein, the term “subject” refers to any animal (e.g., a mammal), including, but not limited to humans, non-human primates, pigs, and the like, which is to be the recipient of a particular treatment.

As used herein, the term “T cell epitope” (also known as antigenic determinant), refers to part of an antigen that is recognized by the immune system, specifically by T cells.

As used herein, the terms “treat”, “treating”, and “treatment” have their ordinary and customary meanings, and include one or more of: blocking, ameliorating, or decreasing in severity and/or frequency a symptom of a disease or condition (e.g., infection by an influenza virus) in a subject. Treatment, e.g., through a vaccine, includes prophylactic or preventative measures that prevent or slow the development of a targeted pathologic condition or disorder.

Influenza A virus (IAV) are classified into H (H1-18) and N (N1-11) subtypes according to antigenic characteristics of the HA and NA surface glycoproteins in the virus. Among various subtypes, H1N1, H1N2, H3N1, and H3N2 are endemic in pigs, while only H1N1 and H3N2 have been found to circulate in humans. Most commercially available influenza vaccines for animals and humans are inactivated influenza vaccines that are typically made by growing target viruses in chicken eggs. Common types of inactivated vaccines that are currently on the market include: whole inactivated virus (WIV) vaccines (often with adjuvant and intramuscularly injected), split virus vaccines, vectored subunit vaccines (delivered either via intramuscular or intranasal means), live attenuated vaccine (via intranasal delivery), and virosome vaccines (see Soema, P. C. et al., Eur. J. Pharm. Biopharm. (2015) 94: 251-263; and Vincent, A L. et al., Vet Microbiol. (2017) 206: 35-44). The overwhelming majority of traditional animal vaccine productions use whole inactivated wild-type viruses, which primarily induces only adaptive antibody response against the HA protein of those wild-type viruses and their antigenically close strains. In additional to poor reliability in egg-based production, traditional inactivated vaccines do not stimulate cellular immune response and work much less well against heterologous influenza infections as the antibody response induced by WIV vaccination does not provide significant protection against antigenically diverse IAV strains (e.g., Rajao, D. and Perez, D., Front. Microbiol. (2018) 9: 123).

Against that background, DNA vaccines have emerged as an attractive alternative with their ability to induce both humoral, and cell mediated or sometimes referred to as “cellular” immune responses (Borggren M., et al., Vaccine 2016; 34: 3634-3640). Humoral immunity refers to immunity mediated by macromolecules found in extracellular fluids such as antibodies, certain antimicrobial peptides and complement proteins, and therefore includes antibody production and accessory processes accompanying it such as cytokine and memory cell production. Aspects of humoral immunity neutralize viruses before host cells are infected or, in the case of non-neutralizing antibodies, may facilitate resistance or contribute to protection.

Meanwhile, cell mediated immunity (CMI) works to prevent an infected individual from prolonged infections, e.g., from microbes like viruses, which could otherwise result in chronic symptoms or death. CMI responses are associated with cells and include the activation of phagocytes, antigen-specific cytotoxic T-lymphocytes, and the release of cytokines. An important part of the CMI involves antigen-presenting cells (macrophages, dendritic cells and B cells) loading antigenic peptides onto its major histocompatibility complex (MHC), which in turn, presents the peptide to receptors on T cells.

In contrast to antibody-centered humoral immunity, CMI responses to epitopes conserved across IAV strains have been found to provide a vaccinated host with broad protection against a multitude of influenza strains. Mouse and human studies have shown that CMI responses to conserved non-structural proteins can be cross-reactive and protect against a variety of IAV subtypes (Lee L Y-H et al., J Clin Invest. (2008) 118: 3478-3490; Price G E et al., Vaccine (2009) 27: 6512-6521). Both CD4+T helper cells and CD8+ cytotoxic T cells contribute to the clearance of IAV from an infected host. Further, memory T cell response improves vaccine efficacy against emerging IAV strains in the presence of cross-reactive T helper cells from prior infection or vaccination. Because adaptive CMI response depends on T cell receptor recognition of peptides bound to MHC molecules presented on cell surface, immunoinformatics tools have been used to accelerate the discovery of T cell epitope for the design of epitope-driven vaccines (EDV) for, e.g., human IAV (Moise L et al., Hum Vaccines Immunother. (2013) 9: 1598-1607).

The combination of both humoral immunity and CMI induced by DNA vaccines offers the hope of broad-spectrum protection to a vaccinated host (e.g., Wei H., et al., Viral Immunol. 2014; 27(1): 14-19). However, there are potential pitfalls that can cause a DNA vaccine to fail, such as: use of low numbers of epitopes, poor epitope sequence conservation among viral strains, poor matching with leukocypte antigen populations, inefficient delivery, and epitopes activating regulatory T cell responses (Moise L et al., Hum Vaccin Immunother. (2015) 11: 2312-2321). Moreover, the lack of quantitative MHC binding data has hindered the use of the same immunoinformatics-based EDV design tools for food animal species such as swine and cattle.

To overcome these issues and to determine which epitopes will best provide protection from swine viral infections, an in silico epitope prediction tool was developed for pigs using an algorithm called “PigMatrix” that is based on the pocket profile method to calculate swine leukocyte antigen peptide binding preferences (Gutierrez A H et al., BMC Bioinformatics (2015) 16: 290). PigMatrix was used to identify class I and II T cell epitopes highly conserved in multiple representative strains of IAV in U.S. swine and predicted to bind to Swine Leukocyte Antigen (SLA) alleles prevalent in commercial swine. Crystal structures of SLA or best-matched Human Leukocyte Antigen (HLA) supertype alleles were used to define potential contact residues involved in binding pockets for class I and II SLA alleles, respectively. Alleles were selected based on prior data indicating their prevalence in outbred swine populations. The resulting, potentially cross-conserved, immunogenic T cell epitopes led to the development of an epitope driven vaccine (EDV) against swine IAV, which vaccine was validated in a swine influenza model (Gutierrez A H et al., PLoS ONE (2016) 11(7): e0159237). Specifically, intramuscular injections of the EDV in young growing pigs showed that the DNA vaccine composed of cross-conserved, immunogenic T cell epitopes stimulated T cell responses reactive to a whole influenza A virus, potentially providing heterologous cross-protection.

Further to the above in vivo validation, and according to an aspect of the present invention, new regimens of the EDV vaccine (prime boost combinations) were tested. According to a further aspect of the present invention, a novel route of administration, namely, intra-dermal injection, of a swine IAV vaccine was also tested.

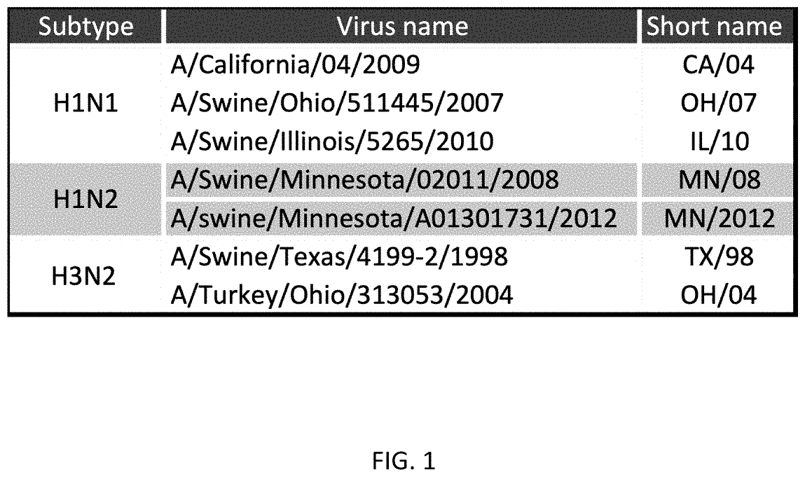

Accordingly, the present invention provides a novel swine influenza vaccine package that includes part compositions for an epitope driven vaccine against swine influenza, preferably multiple strains thereof. Gene sequences of proteins expressed by seven representative and circulating swine IAV strains (FIG. 1) were accessed and downloaded from the Influenza Virus Resource, specifically, the Influenza Virus Sequence Database, at the National Center for Biotechnology Information (NCBI). Of the seven strains, three were H1N1, two H1N2, and two H3N2. In particular, gene sequences for the following external and internal proteins were examined: hemagglutinin (HA), neuraminidase (NA), matrix proteins (M), nucleoprotein (NP), non-structural proteins (NS), RNA polymerase protein A (PA), and RNA polymerase protein B (PB). Potentially cross-conserved peptides around 9 amino acids in length were searched for as candidates for the class I epitope among the seven IAV strains using Conservatrix algorithm (De Groot A S et al., Vaccine (2003) 21: 4486-4504) as 9-mers fit well in class I SLA alleles' binding grooves. Similarly, peptides around 15 amino acids in length (e.g., 13-17 a.a.) were searched for as candidate epitopes for the class II SLA alleles using the same Conservatrix algorithm (EpiVax, Providence, R.I.).

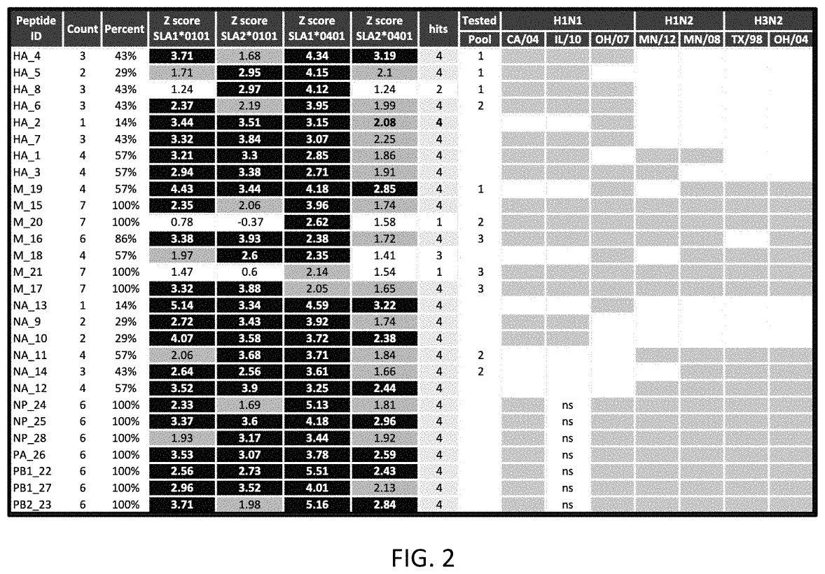

The aforementioned immunoinformatics-based EDV design tool, PigMatrix, was then used to map candidate T-cell epitopes resulting from the Conservatrix analyses across class I and II SLA alleles. Specifically, using PigMatrix's matrix-based algorithm, candidate epitopes were scored for binding potential against a panel of four class I SLA alleles (SLA-1*0101, 1*0401, 2*0101, and 2*0401) (FIG. 2). PigMatrix raw scores were standardized to Z-scores in order to make comparison of each candidate epitope across multiple SLA alleles. Peptides with Z-scores in the tope 5% of the sample 9-mers were identified as potential SLA ligands with a high degree of likelihood. Based on the PigMatrix score, SLA class I allele coverage and IAV strain coverage, 28 candidates were selected as putative SLA class I-restricted epitopes for purpose of the present invention.

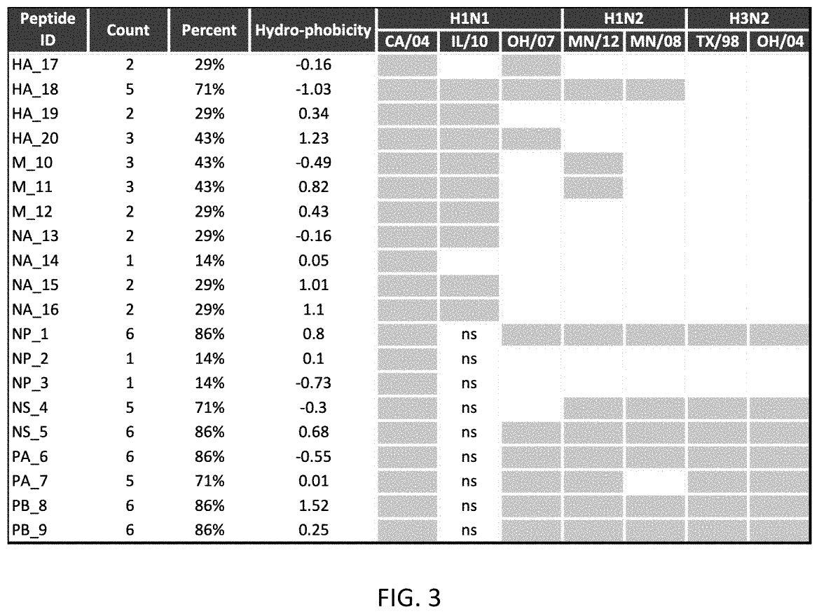

For SLA class II-restricted epitopes, candidates were mapped across a panel of four class II SLA alleles (SLA-DRB1*0101, 0201, 0401, and 0601) using PigMatrix and EpiAssembler programs (EpiVax, Providence, R.I.) (FIG. 3). PigMatrix raw scores were standardized to Z-scores in order to make comparison of each candidate epitope across multiple SLA alleles. Peptides with Z-scores in the tope 5% of the sample 9-mers were identified as potential SLA ligands with a high degree of likelihood. EpiAssembler was used to construct 16-25 amino acid length SLA-DRB1-restricted sequences that were highly conserved in IAV strains, promiscuous (predicted to bind to multiple alleles), and enriched for immunogenicity (immunogenic consensus sequences or ICS). The density of predicted binding motifs in each ICS was scored (i.e. cluster score) using ClustiMer. The cluster score represents the deviation in predicted epitope content from baseline expectation based on random peptides. ICS with cluster scores above 10 were considered to be high-quality clusters for inclusion in the prototype vaccine. Based on the PigMatrix score, SLA class II allele coverage and IAV strain coverage, 20 candidates were selected as putative SLA class II-restricted epitopes for purpose of the present invention.

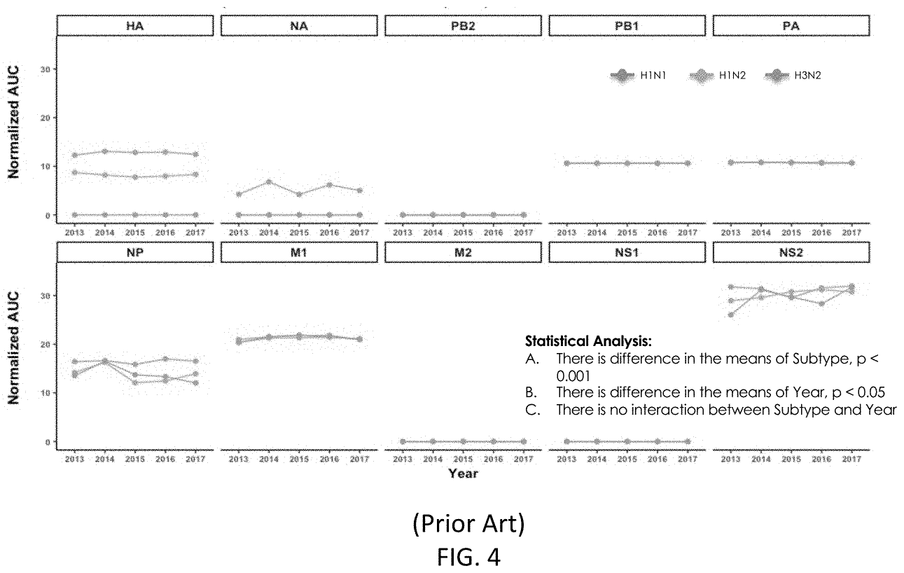

To further determine if the class II T cell epitopes screened according to the above-described process would likely provide protection across a spectrum of IAV strains including future new strains, a computational tool for T-cell epitope content comparison (EpiCC) was employed. EpiCC was developed to make pairwise comparison of protein sequences based on T-cell epitope content in order to evaluate how closely related different strains are, antigenically speaking (Gutierrez A H et al., Influenza Other Respi Viruses (2017) 11: 531-542). EpiCC analyses show that not only there was a high degree of conservation between circulating strains (H1N1, H1N2, and H3N2) and the class II T cell epitopes contained in an EDV previously created in 2013 using a similar PigMatrix-driven process described in the present invention, but also that the conservation was steady at least over the period of 2013 to 2017 (FIG. 4). This provides further analytical support for the cross-protective potential in the EDV component of the present invention across IAV strains and years.

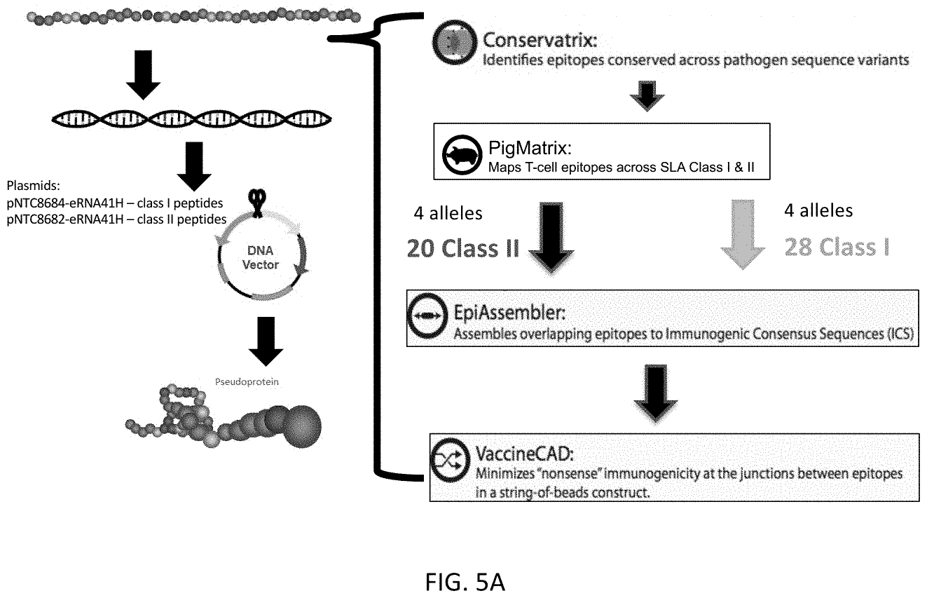

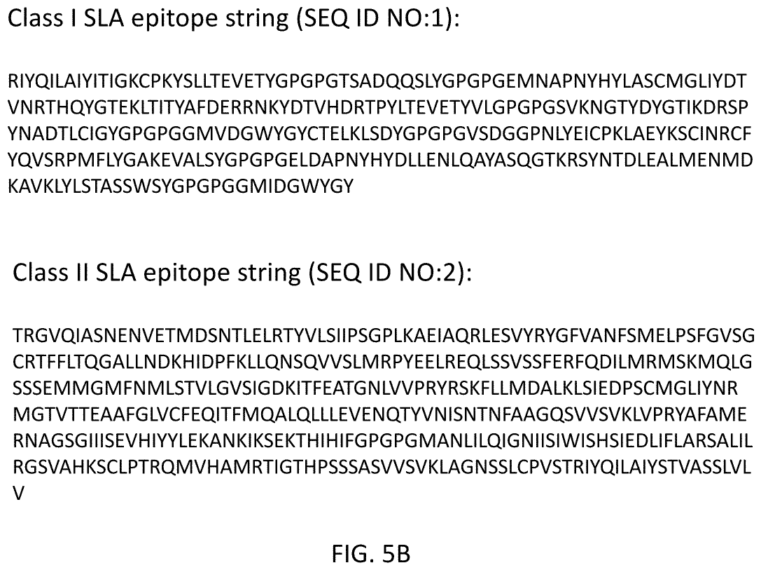

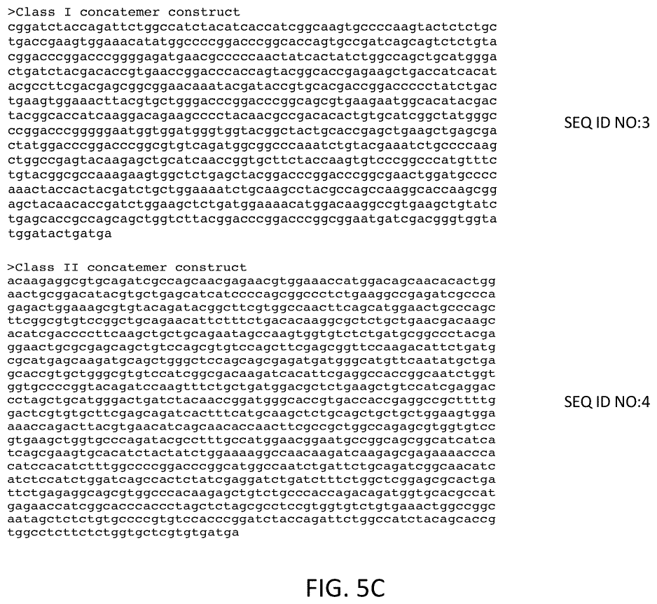

Referring now to the right side of FIG. 5A, once the conserved class I and II T cell epitope candidates were selected based on PigMatrix algorithm, the two classes of epitopes were knit together, respectively within each class, taking into account of any overlaps, to form highly immunogenic consensus sequences (ICS) using another immunoinformatics tool called “EpiAssembler” (EpiVax, Providence, R.I.). Subsequently, another algorithm called “VaccineCAD” (EpiVax, Providence, R.I.) was used to link those epitopes, within each class, into a string-of-beads construct as illustrated on top of the left side of FIG. 5A, while minimizing “nonsense” immunogenicity at the junctions between them to avoid non-specific immune reactions from the final vaccine.

Next, as further illustrated on the left side of FIG. 5A, the linked string-of-beads constructs, e.g., an embodiment that includes amino acid sequences represented by SEQ ID NOS:1 and 2, were reverse-translated into RNA sequences and then further reverse-transcribed into complementary DNA sequences, which, in turn, were pasted into two separate DNA vectors (Nature Technology Corporation, Lincoln, Nebr., USA) as an EDV product. Because multiple triplets of DNA code can eventually translate to the same amino acid residue, a polypeptide sequence such as SEQ ID NO:1 or 2 (FIG. 5B) can be generated using multiple DNA sequences that, for purpose of the present invention, are functional equivalents of each other as long as they all give rise to the same or substantially the same amino acid sequence. In one embodiment of the invention, an EDV is provided containing plasmid DNAs (pDNAs) that carry SEQ ID NOS:3 and 4 (FIG. 5C), e.g., on separately vectors, as SEQ ID NOS:3 and 4 translate into SEQ ID NOS:1 and 2, respectively. In a feature, the EDV may include concatemer constructs in its pDNAs, i.e., with repeats of the coding sequence such as one or more of SEQ ID NOS:3 and 4 or their functional equivalents. Embodiments where one or more copies of SEQ ID NOS:3 and 4 or their functional equivalents are carried on the same DNA molecule are also contemplated by the present invention.

Accordingly, the present invention provides, in a preferred embodiment, an epitope-driven vaccine component containing the sequences represented by SEQ ID NOS:3 and 4 or functional equivalents thereof. In aspects, the present disclosure provides a nucleic acid having a sequence comprising, consisting of, or consisting essentially of one or more of SEQ ID NOS: 3 and 4 (and fragments or variants thereof). In aspects, the present disclosure provides a nucleic acid with at least 80%, 85%, 90%, or 95% homology to SEQ ID NO: 3. In aspects, the present disclosure provides a nucleic acid with at least 80%, 85%, 90%, or 95% homology to SEQ ID NO 3, provided said polypeptide encoded by said nucleic retains anti-IAV activity. In aspects, the present disclosure provides a nucleic acid with at least 80%, 85%, 90%, or 95% homology to SEQ ID NO 4. In aspects, the present disclosure provides a nucleic acid with at least 80%, 85%, 90%, or 95% homology to SEQ ID NO 4, provided said polypeptide encoded by said nucleic retains anti-IAV activity. The nucleic acids may be used to produce the one or more polypeptides as described herein in vitro, or to produce cells expressing the polypeptide on their surface, or to produce vaccines wherein the active agent is the nucleic acids or a plasmid or vector containing one or more of the nucleic acids. In aspects, said nucleic acids are DNA or RNA (e.g., mRNA). In aspects, said nucleic acids may be included in a plasmid or vector. By “functional equivalents,” in this context, it is meant to include other genetic materials, e.g., DNA or RNA, naturally occurring, modified or analogs thereof, that could lead to the production, in vitro or in vivo, of polypeptides containing SEQ ID NOS:1 and 2 (respectively) or substantially the same sequences, i.e., polypeptides with at least 80%, 85%, 90%, or 95% homology to SEQ ID NOS:1 and 2.

In aspects, the sequences represented by SEQ ID NOS:3 and 4 or functional equivalents thereof provides a polypeptide with at least 80%, 85%, 90%, or 95% homology to SEQ ID NOS: 1 and 2, respectively. In aspects, the sequences represented by SEQ ID NOS:3 and 4 or functional equivalents thereof provides polypeptides having anti-IAV activity, said polypeptides with at least 80%, 85%, 90%, or 95% homology to SEQ ID NOS: 1 and 2, respectively. As described previously, anti-IAV activity includes, in aspects: being capable of stimulating, inducing, and/or expanding an immune response to IAV (e.g., a cellular or humoral immune response to IAV) and/or associated diseases in a subject; being capable of stimulating, inducing, and/or expanding an IAV-specific IFNγ response (e.g., by lymphocytes such as PMBC, or effector CD4+ and/or CD8+ T-cells), and/or being capable of inducing immunity against IAV in a host subject (e.g., a pig). In aspects, such polypeptides or nucleic acids having anti-IAV activity will reduce the disease symptoms resulting from IAV challenge by at least about 5% to about 50%, at least about 10% to about 60%, at least about 30% to about 70%, at least about 40% to about 80%, or at least about 50% to about 90% or greater, including any value or range therebetween. Again, anti-IAV activity can be determined by various experiments and assays as known to those of skill in the art, including the experiments and assays as disclosed in the Example section herein. In one embodiment, the vaccine is a DNA vaccine containing multiple plasmid DNAs. Nucleotide modifications are well known in the art, and include modifications from naturally occurring forms of a nucleotide residue in one or more of its sugar, backbone, and/or base unit. In an embodiment, a backbone-modified nucleotide has at least a modification in an internucleoside linkage, e.g., to include at least one of a nitrogen or sulphur heteroatom. In some embodiments, the modified internucleoside linkage is or includes: phosphorothioate (P═S) group, phosphotriesters, methylphosphonates, or phosphoramidate.

For nucleic acids of the present disclosure, a “variant” comprises a deletion and/or addition of one or more nucleotides at one or more internal sites within the nucleic acid sequences of the instant disclosure and/or a substitution of one or more nucleotides at one or more sites in the nucleic acid sequences of the instant disclosure. One of skill in the art will recognize that variants of the nucleic acid of the invention will be constructed such that the open reading frame is maintained. For nucleic acid, conservative variants include those sequences that, because of the degeneracy of the genetic code, encode the amino acid sequence of one of the nucleic acids of the instant disclosure. Naturally occurring allelic variants such as these can be identified with the use of well-known molecular biology techniques, as, for example, with polymerase chain reaction (PCR) and hybridization techniques as outlined below. Variant nucleic acids also include synthetically derived nucleic acids, such as those generated, for example, by using site-directed mutagenesis but which still encode a nucleic acid having the desired activity of the instant disclosure (i.e., encoding a polypeptide that possesses the desired biological activity, that is, WIC binding propensity and/or TCR specificity, or anti-IAV activity, as described herein). Generally, variants of a particular nucleic acid of the invention will have at least about 80%, 85%, 90%, 91%, 92%, 93%, 94%, 95%, 96%, 97%, 98%, 99% or more sequence identity to that particular nucleic acid as determined by sequence alignment programs and parameters described elsewhere herein.

Variants of a particular nucleic acid of the instant disclosure (i.e., the reference nucleic acid) can also be evaluated by comparison of the percent sequence identity between the polypeptide encoded by a variant nucleic acid and the polypeptide encoded by the reference nucleic acid. Thus, for example, an isolated nucleic acid that encodes a polypeptide with a given percent sequence identity to a polypeptide of SEQ ID NOS: 1 and 2 (including the noted mutations/modifications) are disclosed. Percent sequence identity between any two polypeptides can be calculated using sequence alignment programs and parameters described elsewhere herein. Where any given pair of nucleic acids of the invention is evaluated by comparison of the percent sequence identity shared by the two polypeptides they encode, the percent sequence identity between the two encoded polypeptides is at least about 80%, 85%, 90%, 91%, 92%, 93%, 94%, 95%, 96%, 97%, 98%, 99% or more sequence identity.

In aspects, the present disclosure also includes fragments of the instantly disclosed nucleic acids. In aspects, the present disclosure also encompasses fragments of the variants of the instantly disclosed nucleic acids. Useful fragments (and fragments of the variants of the nucleic acids described herein) include those that could lead to the production, in vitro or in vivo, of polypeptides comprising, consisting essentially of, or consisting of SEQ ID NOS:1 and 2, or variants and fragments thereof, provided said polypeptides retain one or more of the biological activities, particularly: anti-IAV activity. Biologically active fragments of the instantly disclosed nucleic acids are, for example, about 300, 400, 500, 600, 700, 800, 900, 1000, or 1100 or more nucleotides in length, including any value or range therebetween. Fragments can be discrete (not fused to other nucleotides or nucleic acids) or can be within a larger nucleic acid. Several fragments can be comprised within a single larger nucleic acid.

In aspects, the nucleic acid(s) further comprises, or is contained within, an expression cassette, a plasmid, and expression vector, or recombinant virus, wherein optionally the nucleic acid, or the expression cassette, plasmid, expression vector, or recombinant virus is contained within a cell, optionally a human cell or a non-human cell, and optionally the cell is transformed with the nucleic acid, or the expression cassette, plasmid, expression vector, or recombinant virus. In aspects, the cell can be a mammalian cell, bacterial cell, insect cell, or yeast cell. In aspects, the nucleic acid molecules of the present disclosure can be inserted into vectors and used, for example, as expression vectors or gene therapy vectors. Gene therapy vectors can be delivered to a subject by, e.g., intravenous injection, local administration (U.S. Pat. No. 5,328,470) or by stereotactic injection (Chen S H et al., (1994), Proc Natl Acad Sci USA, 91(8):3054-7, which are herein incorporated by reference in their entirety). Similarly, the nucleic acid molecules of the present disclosure can be inserted into plasmids. The pharmaceutical preparation of the gene therapy vector or plasmid can include the gene therapy vector or plasmid in an acceptable excipient and/or carrier (and may also include an optional adjuvant), or can comprise a slow release matrix in which the gene delivery vehicle is imbedded. Alternatively, where the complete gene delivery vector can be produced intact from recombinant cell s, e.g., retroviral vectors, the pharmaceutical preparation can include one or more cells that produce the gene delivery system. Such pharmaceutical compositions can be included in a container, pack, or dispenser together with instructions for administration.

The nucleic acid of the instant disclosure may be DNAs (including but not limited to cDNA) or RNAs (including but not limited to mRNA), single- or double-stranded. The nucleic acid is typically DNA or RNA (including mRNA). The nucleic acid may be produced by techniques well known in the art, such as synthesis, or cloning, or amplification of the sequence encoding the immunogenic polypeptide; synthesis, or cloning, or amplification of the sequence encoding the cell membrane addressing sequence; ligation of the sequences and their cloning/amplification in appropriate vectors and cells. The nucleic acids provided herein (whether RNAs, DNAs, vectors, viruses or hybrids thereof) that encode in whole or in part one or more polypeptides as described herein can be isolated from a variety of sources, genetically engineered, amplified, synthetically produced, and/or expressed/generated recombinantly. Recombinant polypeptides generated from these nucleic acids can be individually isolated or cloned and tested for a desired activity. Any recombinant expression system can be used, including, e.g., in vitro, bacterial, fungal, mammalian, yeast, insect or plant cell expression systems. In aspects, nucleic acids provided herein are synthesized in vitro by well-known chemical synthesis techniques (as described in, e.g., Adams (1983) J Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066, all of which are herein incorporated by reference in their entirety). Further, techniques for the manipulation of nucleic acids provided herein, such as, e.g., subcloning, labeling probes (e.g., random-primer labeling using Klenow polymerase, nick translation, amplification), sequencing, hybridization and the like are well described in the scientific and patent literature (see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993), all of which are herein incorporated by reference in their entirety).

As used herein, two polypeptides (or a region of the polypeptides) are substantially homologous or identical when the amino acid sequences are at least about 45-55%, typically at least about 70-75%, more typically at least about 80-85%, more typically greater than about 90%, and more typically greater than 95% or more homologous or identical. To determine the percent homology or identity of two amino acid sequences, or of two nucleic acid sequences, the sequences are aligned for optimal comparison purposes (e.g., gaps can be introduced in the sequence of one polypeptide or nucleic acid molecule for optimal alignment with the other polypeptide or nucleic acid molecule). The amino acid residues or nucleotides at corresponding amino acid positions or nucleotide positions are then compared. When a position in one sequence is occupied by the same amino acid residue or nucleotide as the corresponding position in the other sequence, then the molecules are homologous at that position. As is known in the art, the percent identity between the two sequences is a function of the number of identical positions shared by the sequences, taking into account the number of gaps, and the length of each gap, which need to be introduced for optimal alignment of the two sequences. Sequence homology for polypeptides is typically measured using sequence analysis software.

In aspects, the present disclosure also encompasses polypeptides having a lower degree of identity but having sufficient similarity so as to perform one or more of the same functions performed by a polypeptide of the instant disclosure (e.g., a polypeptide having a sequence comprising, consisting of, or consisting essentially of one or more of SEQ ID NOS: 1 and 2 as disclosed herein, or a polypeptide encoded by a nucleic acid molecule of the present disclosure, including a nucleic acid molecule having a sequence comprising, consisting of, or consisting essentially of one of more of SEQ ID NOS 3 and 4). Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu, Met, and Ile; interchange of the hydroxyl residues Ser and Thr, exchange of the acidic residues Asp and Glu, substitution between the amide residues Asn and Gln, exchange of the basic residues His, Lys and Arg and replacements among the aromatic residues Trp, Phe and Tyr. Guidance concerning which amino acid changes are likely to be phenotypically silent are found (Bowie J U et al., (1990), Science, 247(4948):130610, which is herein incorporated by reference in its entirety).

In aspects, a variant polypeptide can differ in amino acid sequence by one or more substitutions, deletions, insertions, inversions, fusions, and truncations or a combination of any of these. Variant polypeptides can be fully functional (e.g., retain MHC binding propensity and/or TCR specificity, and/or retain IAV activity) or can lack function in one or more activities. Fully functional variants typically contain only conservative variation or variation in non-critical residues or in non-critical regions; in this case, typically MHC contact residues providing MHC binding is preserved. Functional variants can also contain substitution of similar amino acids that result in no change or an insignificant change in function (e.g., retain MHC binding propensity and/or TCR specificity, and/or retain IAV activity). In aspects, a variant and/or a homologous polypeptide retains the desired anti-IAV activity of the instant disclosure (e.g.: capable of stimulating, inducing, and/or expanding an immune response to IAV (e.g., a cellular or humoral immune response to IAV) and/or associated diseases in a subject; capable of stimulating, inducing, and/or expanding an IAV-specific IFNγ response (e.g., by lymphocytes such as effector CD4+ and/or CD8+ T-cells), and/or capable of inducing immunity against IAV as described herein). Alternatively, such substitutions can positively or negatively affect function to some degree. Non-functional variants typically contain one or more non-conservative amino acid substitutions, deletions, insertions, inversions, or truncation or a substitution, insertion, inversion, or deletion in a critical residue or critical region; in this case, typically TCR contact residues.

In aspects, the present disclosure also includes fragments of the instantly disclosed polypeptides and concatemeric polypeptides. In aspects, the present disclosure also encompasses fragments of the variants of the instantly disclosed polypeptides and concatemeric polypeptides. Useful fragments (and fragments of the variants of the polypeptides and concatemeric polypeptides described herein) include those that retain one or more of the biological activities, particularly: MHC binding propensity and/or TCR specificity, and/or anti-ASFV activity. Biologically active fragments are, for example, about at least 100, 150, 200, 250, 300, or 350 or more amino acids in length, including any value or range therebetween. Fragments can be discrete (not fused to other amino acids or polypeptides) or can be within a larger polypeptide. Several fragments can be comprised within a single larger polypeptide. In aspects, a fragment designed for expression in a host can have heterologous pre- and pro-polypeptide regions fused to the amino terminus of the polypeptide fragment and an additional region fused to the carboxyl terminus of the fragment.

In aspects, the instantly disclosed polypeptides and concatemeric polypeptides of the present disclosure can include allelic or sequence variants (“mutants”) or analogs thereof, or can include chemical modifications (e.g., pegylation, glycosylation). In aspects, a mutant retains the same function, particularly anti-IAV activity. In aspects, a mutant can provide for enhanced binding to MHC molecules. In aspects, a mutant can lead to enhanced binding to TCRs. In another instance, a mutant can lead to a decrease in binding to MHC molecules and/or TCRs. Also contemplated is a mutant that binds, but does not allow signaling via the TCR.

In-depth analysis shows that the class I and II-restricted epitopes represented by SEQ ID NOS:1 and 2 persist across more than 1000 actual circulating swine IAV strains from 2013 to 2017, whereas commercial swine IAV vaccines developed in 2013 exhibited, in contrast, declining conservation with the circulating IAV strains over the same five-year period (Tan S. et al., Vaccines (2021) 9:468). This further supports a potential use where the vaccine embodiment made according to principles of the present invention is used, by itself, or in combination with a commercial swine flu vaccine to provide protection against influenza when antibodies in the host body are not sufficiently cross-reactive. In the combinatorial use, the vaccine of the present invention can be used as part of a prime-boost regiment.

Further testing was conducted to evaluate the above-described EDV embodiment and found strong cell-mediated immune response from the EDV in swine subjects against challenges from IAV strains including pandemic strains, and overall efficacy against such challenges. Moreover, according to aspects of the present invention, efficacies of different regimens/treatment were evaluated and compared where the vaccine components used in the regimen were either repeated doses of the EDV embodiment (a pDNA vaccine) or a commercial, inactivated-IAV-based vaccine, or a combination of doses of both (prime-boost) as a vaccine package.

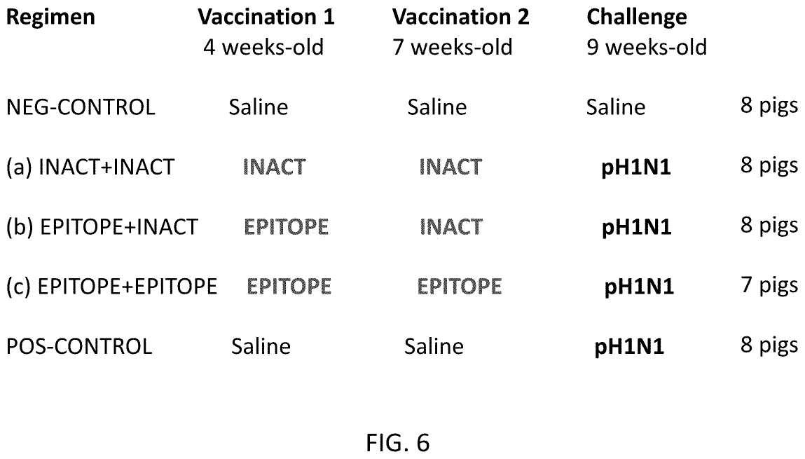

The experiment started with randomly assigning forty IAV-free, young (3-week-old) growing pigs to five different treatment regimens (FIG. 6) designed as follows: besides the two controls, three test regimens involved sequential administrations of vaccines selected from: the above-described, EDV embodiment, namely, a pDNA EDV designed to produce conserved SLA class I and Class II epitopes in the subject (“EPITOPE” in figures) administered through an intradermal means; and a commercially available, inactivated whole virus vaccine, FluSure XP® from Zoetis (“INACT” in figures) administered in an intramuscular fashion. Each test regimen included a first vaccination of the eight IAV-free pigs at 4 weeks of age, followed by a second vaccination of the same pigs at 7 weeks of age (one pig died during the experiment from causes unrelated to the project). At 9 weeks of age, the pigs were either challenged with pH1N1 virus or sham-inoculated in accordance of design of the experiment. Euthanasia and necropsy were conducted at day post challenge (dpc) 5. Specifically, the two control regimens both had unvaccinated pigs (injected with saline solutions) where the negative control was sham-challenged (“NEG CONTROL” in figures) and the positive control was pH1N1-challenged (“POS CONTROL” in figures). For the three test regimens, the sequential administrations of vaccines were, respectively: (a) INACT, and INACT; (b) EPITOPE, and INACT; and (c) EPITOPE, and EPITOPE.

At the time of the H1N1 challenge, all pigs from regimen (a) (INACT-INACT) were IAV seropositive; by dpc 5, all pigs from regimen (b) (EPITOPE-INACT) were IAV seropositive. IFNγ secreting cells, recognizing vaccine epitope-specific peptides and pH1N1 challenge virus were highest in regimen (b) (EPITOPE-INACT) pigs at time of the challenge. Macroscopic lung lesion scores were reduced in all regimen (b) (EPITOPE-INACT) pigs, while regimen (a) (INACT-INACT) pigs exhibited a bimodal distribution of low and high scores akin to naïve challenged animals. Remarkably, no IAV antigen in lung tissues was detected at necropsy in the regimen (b) (EPITOPE-INACT) pigs, indicating complete clearing of any viral infection from at least the lungs under the combination regimen. This demonstrated a unique and unexpected advantage when compared to all other tested regimens where the same kind of vaccines were repeated.

Regimen (b) (EPITOPE-INACT) represents a “prime-boost” approach to vaccination. In a feature of the invention, the “prime-boost” procedure entails a first instance of vaccination that aims to induce both a CMI and humoral responses in the host, followed by a second instance of vaccination that aims to at least strengthen the humoral response. According to the present invention, this can be achieved through administrations of a first DNA vaccine, e.g., a T-cell epitope-driven DNA vaccine, followed by a second protein- or peptide-based vaccine, e.g., a whole inactivated virus vaccine. Examples of the second vaccine further include and are not limited to a split virus vaccine, a subunit vaccine, a live attenuated vaccine, and a virosome vaccine. Our data have supported a heterologous prime-boost regimen in the prevention of swine influenza viral infection (Hewitt J S et al., Vaccine (2019) 37: 4302-4309). In a preferred embodiment of such heterologous prime-boost regimen, an epitope-driven DNA vaccine is followed by a protein- or peptide-based vaccine.

The vaccines of the present disclosure may comprise other ingredients, known per se by one of ordinary skill in the art, such as pharmaceutically acceptable carriers, excipients, diluents, adjuvants, freeze drying stabilizers, wetting or emulsifying agents, pH buffering agents, gelling or viscosity enhancing additives, and preservatives, depending on the route of administration.

Examples of pharmaceutically acceptable carriers, excipients or diluents include, but are not limited to demineralised or distilled water; saline solution; vegetable based oils such as peanut oil, arachis oil, safflower oil, olive oil, cottonseed oil, maize oil, sesame oil, or coconut oil; silicone oils, including polysiloxanes, such as methyl polysiloxane, phenyl polysiloxane and methylphenyl polysolpoxane; volatile silicones; mineral oils such as light liquid paraffin oil, or heavy liquid paraffin oil; squalene; cellulose derivatives such as methylcellulose, ethylcellulose, carboxymethylcellulose, carboxymethylcellulose sodium salt, or hydroxypropyl methylcellulose; lower alkanols, for example ethanol or isopropanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrrolidone; agar; carrageenan; gum tragacanth or gum acacia; and petroleum jelly. Typically, the carrier or carriers will form from 10% to 99.9% by weight of the vaccine composition and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

Examples of adjuvants include, but are not limited to, oil in water emulsions, aluminum hydroxide (alum), immunostimulating complexes, non-ionic block polymers or copolymers, cytokines (like IL-1, IL-2, IL-7, IFN-α, IFN-β, IFN-γ, etc.), saponins, monophosphoryl lipid A (MLA), muramyl dipeptides (MDP) and the like. Other suitable adjuvants include, for example, aluminum potassium sulfate, heat-labile or heat-stable enterotoxin(s) isolated from Escherichia coli, cholera toxin or the B subunit thereof, diphtheria toxin, tetanus toxin, pertussis toxin, Freund's incomplete or complete adjuvant, etc. Toxin-based adjuvants, such as diphtheria toxin, tetanus toxin and pertussis toxin may be inactivated prior to use, for example, by treatment with formaldehyde. Further adjuvants may include, but are not limited to, poly-ICLC, 1018 ISS, aluminum salts, Amplivax, AS 15, BCG, CP-870,893, CpG7909, CyaA, dSLIM, GM-CSF, IC30, IC31, Imiquimod, ImuFact IMP321, IS Patch, ISS, ISCOMATRTX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, and Aquila's QS21 stimulon.

### EXAMPLES

Vaccination:

A DNA vaccine coded “EPITOPE” was prepared as described above and included separate plasmids that respectively included DNA sequences SEQ ID NOS:3 and 4 for producing in vivo polypeptides of SEQ ID NOS:1 and 2. The EPITOPE vaccine was used to vaccinate the pigs in the (b) EPITOPE-EPITOPE and (c) EPITOPE-INACT groups (FIG. 6). Specifically, the EPITOPE vaccine was composed of a 1:1 mixture of two plasmids: one carries a synthetic gene encoding 28 SLA class I epitopes targeted to the proteasome by an N-terminal ubiquitin fusion for endogenous antigen processing (SEQ ID NO:3); the other plasmid carries a genetic code for 20 SLA class II epitopes targeted for secretion by a tissue plasminogen activator signal sequence for processing via the exogenous pathway (SEQ ID NO:4). Downstream IAV epitope products were built into the EPITOPE vaccine for representation by prevalent SLA alleles, and because the epitopes were highly conserved in circulating strains of swine IAV.

High-purity plasmids for immunizations were prepared at research grade (Nature Technology; Lincoln, Nebr., USA). All pigs in the EPITOPE-EPITOPE group were vaccinated with the EPITOPE vaccine at 4 and 7 weeks of age and all EPITOPE-INACT pigs were vaccinated at 4 weeks of age with this vaccine. The DNA vaccine was prepared using 2 mg/mL plasmid DNA (pDNA) in Tris-EDTA buffer (10 mMTris pH 8.0, 1 mM EDTA) diluted to 2661 g/mL with phosphate buffered saline (PBS). The pDNA vaccine (0.5 mL dose containing 133 lg of the plasmid) was administered intradermally in the neck using a commercial needle-free high-pressure device (Pulse 50™ Micro Dose Injection System, Pulse NeedleFree Systems; Lenexa, Kans., USA) set at 65 lb per square inch (PSI).

DNA vaccines such as the embodiment of the invention can also be administered by any other means known to one skilled in the art, e.g., intramuscularly via gene gun or electroporation. Administering the vaccine via the intradermal route, however, has advantages in mass vaccination of pigs under field conditions due to the ease of using needle-free devices and avoidance of problems with broken needles. Moreover, the intradermal injection avails the administrator to effectiveness of dermal dendritic cells in antigen capture and T lymphocyte presentation, whereas muscle tissue has many fewer antigen presenting cells.

A commercially available, inactivated whole IAV-S vaccine (Flu-Sure XP®, Zoetis; Lot 275030; Parsippany, N.J., USA) was administered as the “INACT” vaccine in this example at 4 and 7 weeks of age to INACT-INACT pigs and at 7 weeks to the EPITOPE-INACT pigs. Manufacturer instructions were followed, and 2 mL of the “INACT” vaccine was administered intramuscularly into the neck area of each pig. The “INACT” vaccine contained H3N2 Cluster IV-A&B, H1N2 Delta-1, and H1N1 Gamma IAV strains but did not contain pH1N1 antigens.

IAV Challenge

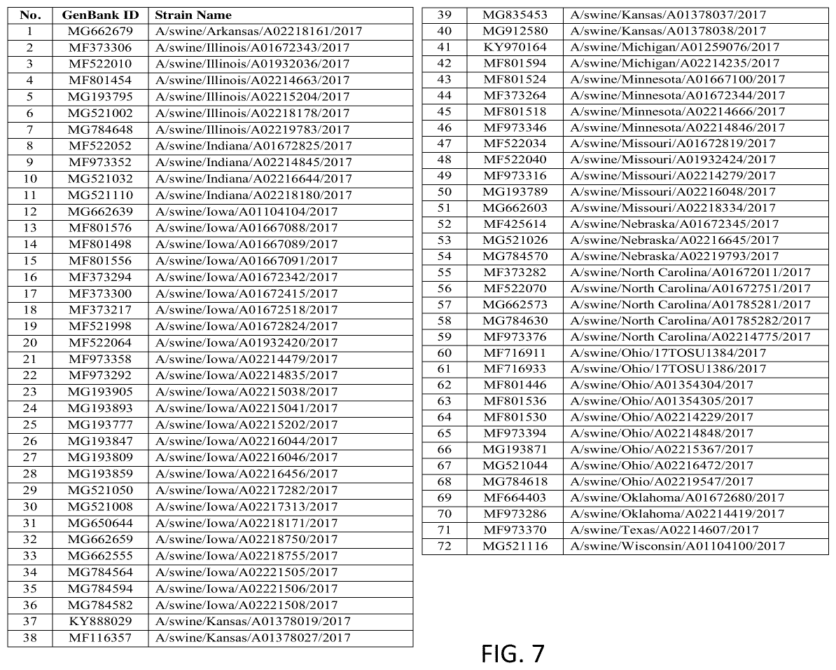

The IAV challenge strain used in this example, pH1N1 strain A/swine/Iowa/A01104104/2017, was selected using immunoinformatics analysis from among 72 swine IAV pH1N1 2017 isolates for which whole genomes were available (FIG. 7) for closest T cell epitope relatedness to the pDNA vaccine. Two validated immunoinformatic algorithms were applied: T cell epitope content comparison (EpiCC) (Gutierrez A H et al., Influenza Other Respir Viruses (2017) 11: 531-542), and JanusMatrix (JMX) (Moise L et al., Hum Vaccin Immunother (2013) 9: 1577-1586). Pairwise comparisons between the EPITOPE vaccine and 72 circulating strains were conducted using EpiCC to analyze overall vaccine epitope cross-conservation on an antigen-by-antigen basis (data not shown). Higher EpiCC scores are associated with greater T cell epitope relatedness between the EPITOPE-IAV vaccine and circulating strains. EpiCC scores were summed over all IAV antigens per SLA class and ranked for challenge strain selection (data not shown).

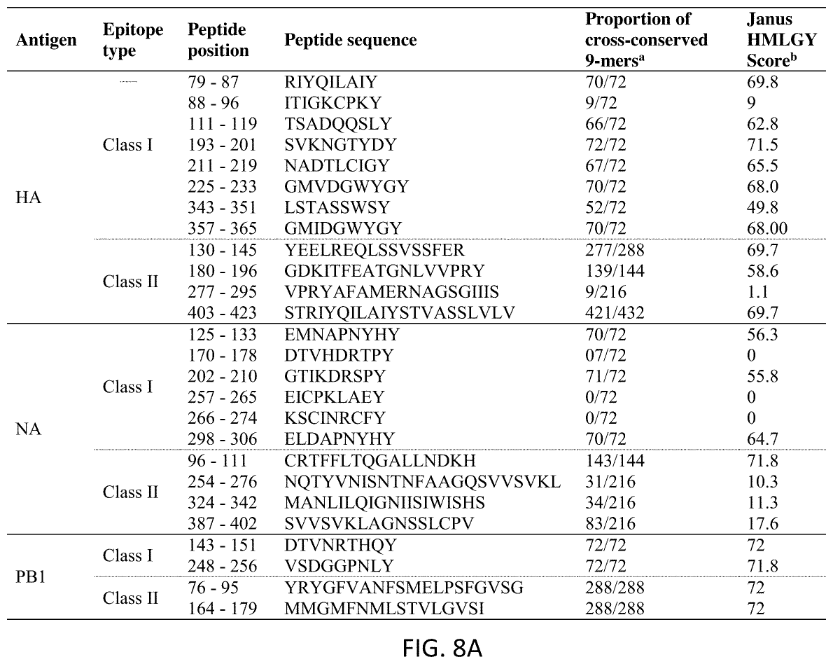

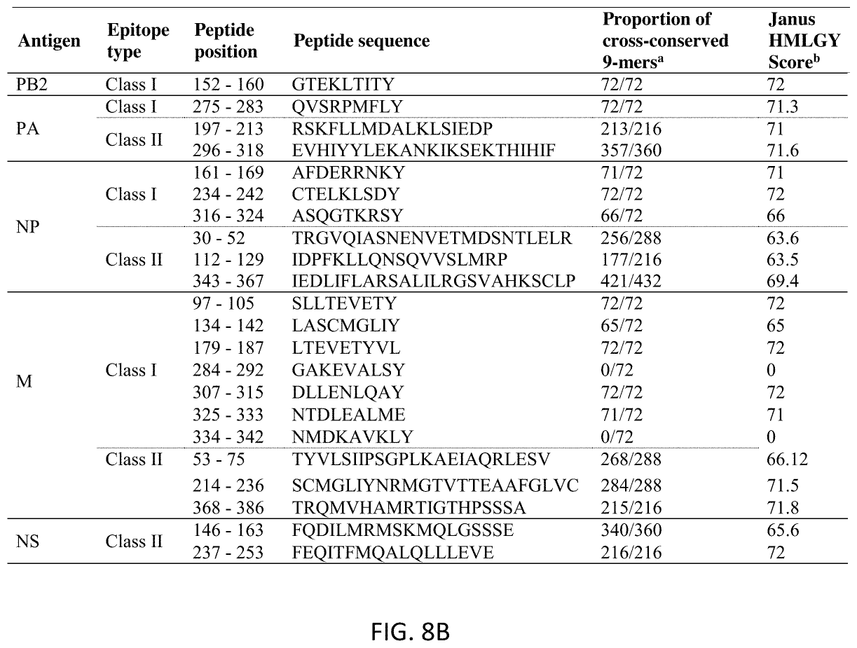

As a complementary approach, JanusMatrix (JMX) (EpiVax, Providence, R.I.) was used to analyze sequences on an epitope-by-epitope basis to identify 48 identical T cell epitopes in the EPITOPE vaccine among the set of 72 H1N1 strains circulating in 2017 (FIGS. 8A and 8B). JMX calculates the Janus Homology Score, which represents the average depth of coverage in the search database of circulating strains for each Epi-Matrix hit in the input vaccine sequence. It considers all constituent 9-mers in any given peptide, including flanks. Strains showing the highest JMX matched SLA class I and II epitopes were ranked for challenge strain selection (data not shown).

The pH1N1 challenge strain A/swine/Iowa/A01104104/2017 was purchased through the National Veterinary Services Laboratories and the USDA swine surveillance system. For the challenge, the pigs were anaesthetized using a ketamine (8 mg/kg), xylazine (4 mg/kg), and telazol (6 mg/kg) combination as described in Rajao D S et al., Vaccine (2014) 32: 5170-5176. Each pig was inoculated with the pH1N1 by administering 2 mL intratracheally and 1 mL intranasally for a total of 3×10^{5.1} 50% tissue culture infectious dose (TCID₅₀) per pig. NEG-CONTROL pigs were similarly inoculated with saline.

Post-Vaccination Humoral/Antibody Response

Serum antibody levels against IAV under all five test regimens (FIG. 6) were measured using a commercial blocking ELISA kit (Swine Influenza Virus Antibody Test Kit, IDEXX Laboratories, Inc., Westbrook, Me., USA) based on detecting antibodies against the IAV nucleoprotein (NP), as per manufacturer's instructions. A sample to negative (S/N) ratio ≥0.60 was considered antibody negative.

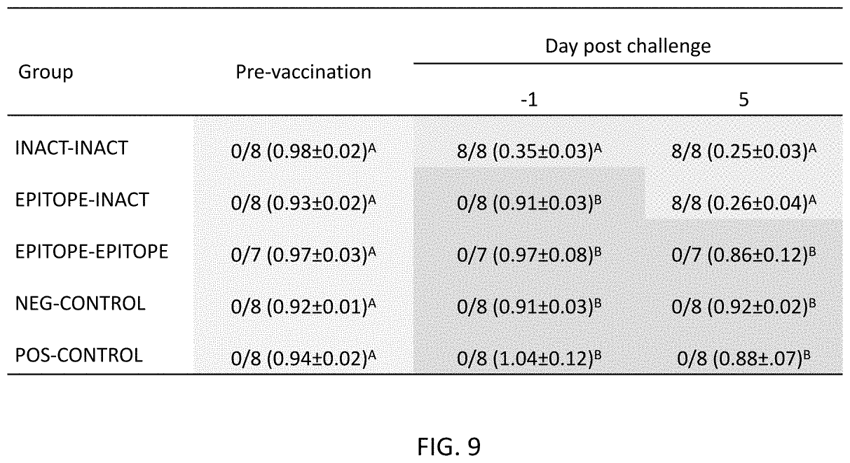

The mean group IAV NP ELISA S/N ratios are summarized in FIG. 9, which shows only pigs receiving INACT vaccine developed antibodies against IAV within five days after the IAV infection, whether they received one or two doses. At dpc-1, all INACT-INACT pigs (regimen (a)) had detectable IAV antibodies and by dpc 5, all pigs in the INACT-INACT and the EPITOPE-INACT (regimen (b)) groups were seropositive. None of the other pigs seroconverted over the duration of the study. In other words, during the period tested, no anti-IAV antibodies were detected in pigs that received the epitope-driven DNA vaccine unless they had also received a dose of the more traditional, viral-protein-based vaccine, e.g., one that is based on an inactivated whole virus. This does not mean, of course, that no detectable amount of IAV-specific antibodies would develop in the host subjects receiving just the EPITOPE vaccine, but that within fives days after infection, the INACT vaccine showed a faster antibody response.

Post-Vaccination CMI Response

Peripheral blood mononuclear cells (PBMCs) collected on dpc 0 were tested for the presence of a CMI response using a commercial IFNγ enzyme-linked immunospot (ELISpot) assay kit (Porcine IFN-gamma ELISpot kit, R&D Systems Inc, Minneapolis, Minn., USA) as per the manufacturer's directions. To each well, 50 lL of complete RPMI was added to pre-wet the membranes, as suggested in literature. A total of 2.5×10⁵ viable PBMC in 100 μL of complete RPMI 1640 media supplemented with 10% heat-inactivated fetal bovine serum were seeded into pretreated microplates (provided in the kit). The seeded cells were stimulated with either the challenge pH1N1 at a concentration of 2.5×10⁵ TCID₅₀ to produce a multiplicity of infection of 1 or pooled peptides at a concentration of 2 μg in 100 μL per well. The peptides were selected to match the epitopes presented in the pDNA vaccine which included sequences derived from influenza structural and nonstructural proteins. For control purposes, PBMCs were stimulated with 0.25 μg pokeweed mitogen (MP Biomedicals™, Santa Ana, Calif., USA) in 100 μL of complete RPMI. The cells were then incubated for 36 hours at 37° C. in a 5% CO2 incubator. Subsequently, the ELISPOT assay was performed according to the manufacturer's instructions. Blue-black colored precipitate spots corresponding to activated IFNγ secreting cells were counted with an ELISPOT reader (ImmunoSpot ELISPOT analyzer, Cellular Technology Limited, Cleveland, Ohio, USA).

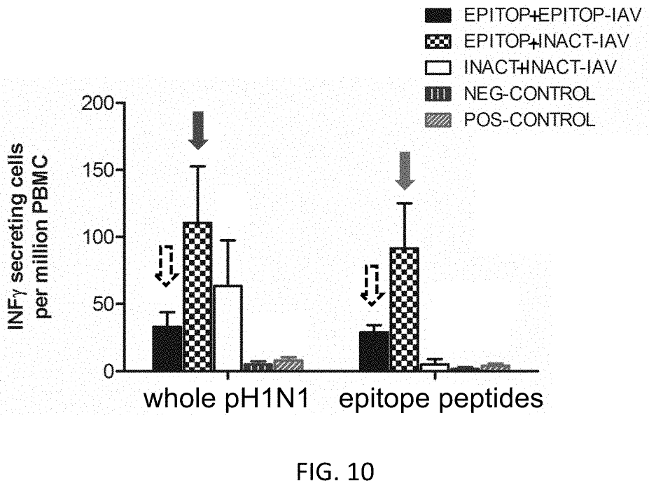

Results of IFNγ secreting cells detected in PBMC a day before the IAV challenge as an indication of the CMI response under different regimens are shown in FIG. 10. Side-by-side comparisons were made of pigs undergoing three different vaccination regimens (FIG. 6): (a) two sequential doses, three weeks apart, of a commercial vaccine based on inactivated viral proteins (INACT-INACT), (b) a single dose of the epitope DNA vaccine followed by a single dose of the inactivated-virus-based vaccine in three weeks (EPITOPE-INACT), and (c) two sequential doses, again three weeks apart, of an epitope DNA vaccine (EPITOPE-EPITOPE). Regimens (a) and (c) can be viewed as homologous prime-boost treatment, while regimen (b) a heterologous, or combinational, prime-boost treatment. All of the vaccine regimens were followed by a challenge with a pH1N1 strain and were compared to each other and to non-vaccinated but pH1N1 challenged pigs (POS-CONTROL).

Results in FIG. 10 lead to a few significant findings. First, while the traditional viral-protein-based vaccine was able to produce IFNγ secreting cells in pigs against the whole pH1N1 virus, it failed to elicit any significant CMI response against epitope peptides of various IAV strains. In contrast, any regimen that included at least one dose of the pDNA vaccine embodiment of the invention (the EPITOPE-INACT or EPITOPE-EPITOPE regimen) showed strong CMI responses against both epitope peptides of various IAV strains as well as the whole pH1N1 virus, suggesting strong efficacy in terms of protection against a wide spectrum of IAV strains. Second, these CMI recall responses were further boosted in pigs vaccinated in the heterologous prime-boost regimen (EPITOPE-INACT) compared to pigs vaccinated with the homologous prime-boost regimen using the same DNA vaccines twice (EPITOPE-EPITOPE) against both the EPITOPE vaccine peptides and the whole pH1N1 virus. In both cases, this boosting effect more than doubled the amount of IFNγ-producing cells detected compared to the EPITOPE-EPITOPE regimen, catapulting the heterologous prime-boost regimen (EPITOPE-INACT) into the strongest recall response facing both the pH1N1 and epitope peptide challenges. In other words, embodiments of the heterologous prime-boost regimen of the invention produced the strongest CMI immune response that was effective against multiple IAV strains. The great prospects for the pDNA vaccine embodiment, either administered by itself or in combination with a viral-protein-based vaccine, were further validated by the clinical results described below.

Clinical Disease Signs

No clinical signs of respiratory diseases were observed in any of the pigs before the IAV challenge and were never observed in any of the NEG-CONTROL pigs. The average daily weight gain (in g±SEM) of the pigs between the time of pH1N1 challenge and the necropsy was 631.8±20.3 for the NEG-CONTROL group, 558.9±64.7 for the INACT-INACT group, 526.5±37.6 for the EPITOPE-INACT group, 601.7±27.3 for the EPITOPE-EPITOPE group, and 502.2±26.7 for the POS-CONTROL group. Here, the groups were not significantly different from each other (P=0.14).

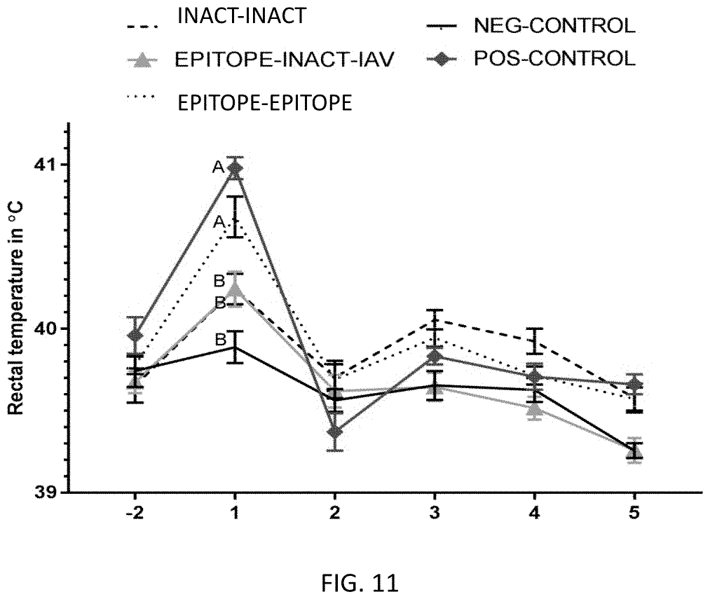

All of the sample pigs' rectal temperatures after IAV challenge are summarized in FIG. 11. Rectal temperatures spiked 24 hours after challenge in all IAV challenged pig groups: 1/8 (one out of eight) INACT-INACT, 1/8 EPITOPE-INACT pigs, 5/7 EPITOPE-EPITOPE pigs, and 8/8 POS-CONTROL pigs had temperatures above 40.5° C. at dpc 1. A sporadic cough was first recognized between dpc 1-3 in the different treatment groups and became persistent by dpc 3-5 in individual IAV infected pigs across all treatments. The average length of coughing in a pig was 1.6±0.7 days in INACT-INACT pigs, 0.6±0.3 days in EPITOPE-INACT pig, 1.4±0.7 days in EPITOPE-EPITOPE pigs, and 0.4±0.2 days in the POS-CONTROL-IAV pigs. Nasal discharge was noted as watery and present in both nostrils at least once in 5/8 INACT-INACT pigs, 2/8 EPITOPE-INACT pigs, 1/7 EPITOPE-EPITOPE pigs, and 4/8 POS-CONTROL pigs.

Compared to the biggest spike in temperature (a clinical symptom of IVA) on dpc 1, administration of the DNA vaccine alone (EPITOPE-EPITOPE) did alleviate the fever, but the effect was not as marked as the combination regimen (EPITOPE-INACT). Moreover, the heterologous prime-boost regimen (EPITOPE-INACT) showed the largest effect among tested vaccine regimens in reducing the body temperature of the vaccinated pig pretty much throughout the post challenge period when temperatures were taken i.e., on dpc 1, 3, 4, and 5.

Clinical Results-IAV RNA Shedding

The overall shedding results were significant (P<0.001) for treatment, dpc, and treatment within dpc. All nasal swabs obtained from the NEG-CONTROL pigs were negative for the presence of IAV RNA (data not shown). In contrast, apart from one INACT-INACT pig that was IAV RNA negative on dpc 1 and another INACT-INACT pig that was negative on dpc 1, 2 and 3, all nasal swabs from IAV infected pigs were IAV RNA positive regardless of treatment regimen.

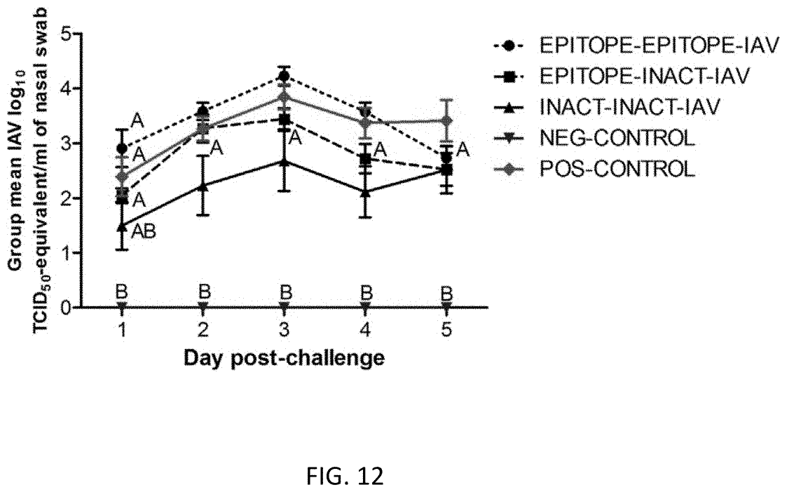

The INACT vaccine appeared to be more effective than the EPITOPE vaccine in reducing viral shedding during the early days post challenge (e.g., the first four days dpc in this example), while the combination regimen (EPITOPE-INACT) showed an intermediary efficacy in reducing IAV's viral RNA shedding compared to the other two vaccine regimens (FIG. 12). By dpc 5, however, all IAV infected pigs under the three vaccine treatment regimens (the combination, and the two repeat vaccination regimens) had similar IAV RNA shedding, and all at a lower level than the POS-CONTROL. This indicates that both INACT and EPITOPE vaccines were able to reduce viral shedding, but the INACT vaccine appeared to work faster than the EPITOPE vaccine in that regard. This further suggests a potential advantage in the combination or heterologous prime-boost treatment regimen where the early onset benefit from a traditional viral-protein-based vaccine is included in the treatment.

Clinical Results-Lung Lesions and IAV Antigen in Tissue Sections

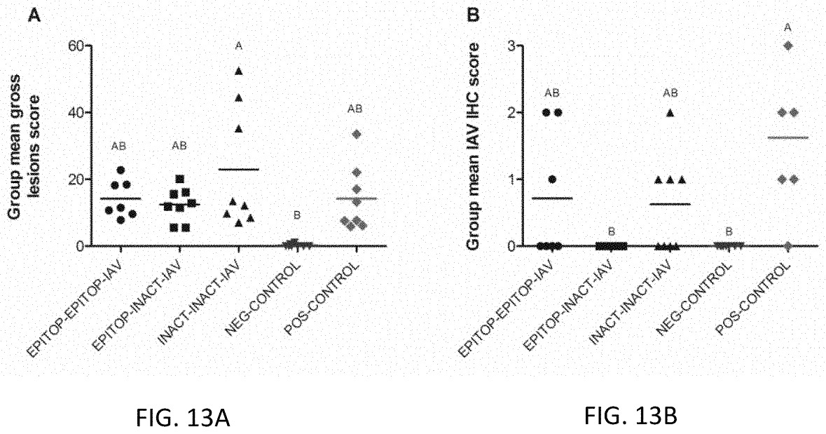

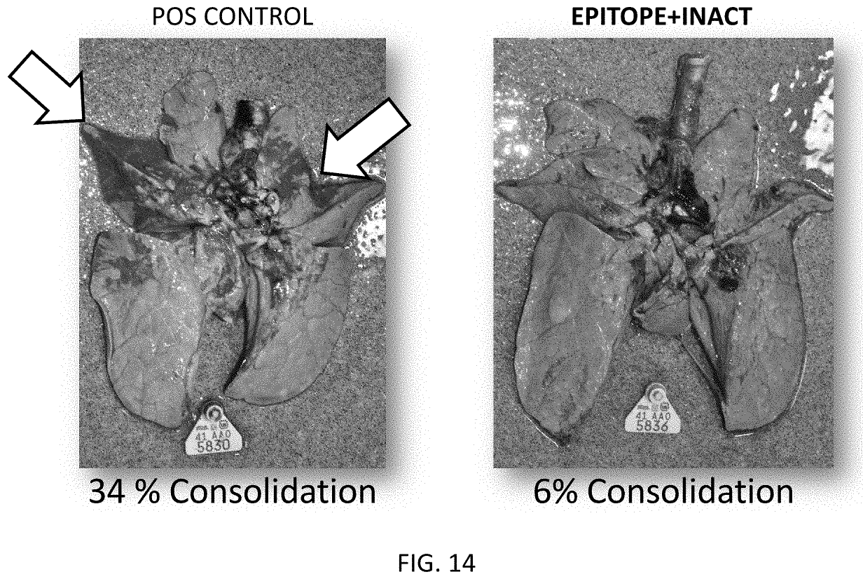

Data on macroscopic lung lesions found in tested pigs are summarized in FIG. 13A. Lesions ranged from moderate to severe, and were characterized as cranioventral, red to purple consolidation that ranged from a checkerboard or lobular pattern to involving the entire cranioventral lobe. Consolidation extended into the cranial portion of the caudodorsal lung lobe in some pigs. INACT-INACT pigs (mean score±SEM, 22.9±6.5) had significantly higher lesion scores compared to the NEG-CONTROL pigs (0.2±0.1), and it was observed that the INACT vaccine treatment alone might lead to a greater degree of lung lesions in some pigs. In contrast, the EPITOPE-INACT (12.4±1.8) and the EPITOPE-EPITOPE (14.2±2.1) groups had many fewer lesions than the INACT-INACT group. Of note, the distribution of pigs with more severe lung lesions was wider in the INACT-INACT and the POS-CONTROL group compared to all other groups and appeared to be bimodal in the INACT-INACT group. In general, treatment involving at least one dose of EPITOPE vaccine was more effective at preventing or reducing macroscopic lung lesions (FIG. 14).

Microscopically, most pig lungs had focal to diffuse and mild to severe necrotizing bronchiolitis, as well as mild to severe peribronchiolar accumulation of inflammatory cells. IAV antigen was demonstrated by IHC stains (FIG. 13B) in all treatment groups except NEG-CONTROL pigs and, surprisingly, EPITOPE-INACT pigs. This shows that while treatment with either EPITOPE or INACT vaccines alone could clear IAV from the lung tissue after the infection, the combination regimen is particularly effective at this, as the pathological result from the heterologous prime-boost treatment was amazingly similar to naïve unchallenged pigs.

The variable genetic nature of influenza virus calls for “universal” vaccine strategies to offer boarder protection against a spectrum of IAV strains. In the above exemplary tests, both immunogenicity and efficacy in the context of homologous (EPITOPE-EPITOPE) and heterologous (EPITOPE-INACT) prime boost regimens were investigated.

Together, these test data indicate that the conserved-epitope-driven DNA vaccine of the invention (EPITOPE), especially in a combination/heterologous prime-boost regimen, is an effective IAV vaccine effective against a wide array of IAV strains including and not limited to H1N1, H1N2, H3N2, and potential emerging strains that have evolved through mutations in existing circulating strains. Strong immune responses against IAV challenges indicating both humoral and CMI responses were detected from pigs inoculated with treatment regimens that included the DNA vaccine of the invention, specifically:

1. At least by dpc 5, pigs receiving the combinational, heterologous prime-boost vaccination regimen had seroconverted and developed IAV-specific antibodies.

2. The DNA vaccine of the invention showed strong CMI response whether administered in a homologous prime-boost regimen (EPITOPE-EPITOPE) or a heterologous prime-boost regimen (EPITOPE-INACT). Moreover, the latter regimen produced far more amount of IFNγ-producing cells than any other regimens, indicating that the DNA vaccine component primed a super strong CMI response with a follow-up dose of viral-protein-based vaccine in a heterologous prime-boost treatment regimen. This is consistent with the observed trend of lower degree of lung lesions and lower antigen levels in lung found in regimens involving one or two doses of the DNA vaccine of the invention.

3. The DNA vaccine of the invention reduced body temperature in inoculated pigs when combined with a viral-protein-based vaccine: body temperature was similar (1 dpc) or lower (3-5 dpc) than the homologous prime-boost regimen that is solely based on the latter vaccine (INACT-INACT).

4. When combined with a viral-protein-based vaccine in a heterologous prime-boost treatment regimen, the DNA vaccine of the invention reduced IAV shedding from the upper respiratory tract: IAV shedding was statistically similar to the INACT-INACT group, and consistent with the observed trend of lower degree of lung lesions and lower antigen levels in lung tissue similar to those found in healthy individuals (NEG-CONTROL).

5. The DNA vaccine of the invention may help improve average daily gain (ADG), as the EPITOPE-EPITOPE group trended for higher ADG.

DNA vaccines are an advantageous vaccination platform due to their ease of production, long shelf stability, and potential for rapidly incorporating precise vaccination targets on demand. In the present invention, the DNA vaccine specifically incorporated genetic codes for conserved T cell epitopes of structural and non-structural proteins that are across multiple IAV strains and subtypes and predicted to have good binding profiles to the SLA class I and class II alleles. Our invention also discovered that the heterologous prime-boosting approach in the EPITOPE-INACT group improved recall responses to EPITOPE vaccine peptides and pH1N1 challenge virus over homologous prime-boost regimen solely based on either EPITOPE or INACT vaccine, showing an additive increase in CMI and a rapid increase in nucleoprotein-specific antibody levels upon challenge, as well as good clinical protection. The data are indicative of some degree of an enhanced cytotoxic T-cell response, and humoral response on day 5 after IAV infection was similar to the INACT vaccine group. Furthermore, the lung IAV antigen levels were much reduced for the heterologous prime-boost regimen. Accordingly, the preferred embodiment of the present invention is a heterologous prime-boosting regimen where both the DNA vaccine of the invention and a more traditional protein-based vaccine are used.

While the present invention has been particularly shown and described with reference to the structure and methods disclosed herein and as illustrated in the drawings, it is not confined to the details set forth and this invention is intended to cover any modifications and changes as may come within the scope and spirit of the following claims. All publications and patent literature described herein are incorporated by reference in entirety to the extent permitted by applicable laws and regulations.

1. A swine influenza vaccine package comprising a first vaccine and a second vaccine, wherein:
a. said first vaccine comprises nucleic acid molecules that lead to the production, in vitro or in vivo, of polypeptides sharing at least 80% homology with at least one of SEQ ID NO:1 and SEQ ID NO:2; and
b. said second vaccine comprises an inactivated swine influenza vaccine.

2. The vaccine package of claim 1, wherein said nucleic acid molecules comprise one or more DNA molecules comprising a sequence that shares at least 80% homology with at least one of SEQ ID NO:3 and SEQ ID NO:4 or a functional equivalent thereof.

3. The vaccine package of claim 2, wherein the DNA molecules consist of naturally occurring deoxyribonucleotide residues.

4. The vaccine package of claim 2, wherein at least one of the DNA molecules comprises a modified deoxyribonucleotide residue.

5. The vaccine package of claim 1, effective in eliciting immunity against one or more influenza viral strains.

6. The vaccine package of claim 5, wherein said one or more influenza strains are selected from the group consisting of H1N1, H1N2, and H3N2.

7. The vaccine package of claim 1, for use in a pig.

8. The vaccine package of claim 1, formulated and packaged for intradermal administration.

9. The vaccine package of claim 1, further comprising an adjuvant.

10. The vaccine package of claim 1 wherein said second vaccine comprises a whole inactivated virus vaccine.

11. The vaccine package of claim 1 wherein said second vaccine is selected from the group consisting of a split virus vaccine, a subunit vaccine, a live attenuated vaccine, and a virosome vaccine.

12. A method of inoculating a pig against swine influenza virus, the method comprising administering to a pig facing potential exposure to one or more influenza strains an immunogenically effective amount of a first nucleic acid vaccine and an immunogenically effective amount of a second swine influenza vaccine separated by an interdose interval, wherein said first vaccine comprises one or more nucleic acid molecules, the sequence of which lead to the production in the pig of polypeptides sharing at least 80% homology with at least one of SEQ ID NO:1 and SEQ ID NO:2.

13. The method of claim 12 wherein said first nucleic acid vaccine is administered first.

14. The method of claim 12 wherein said second swine influenza vaccine is administered first.

15. The method of claim 12 wherein the interdose interval is at least 24 hours.

16. (canceled)

17. (canceled)

18. The method of claim 12, wherein said nucleic acid molecules comprise one or more DNA molecules comprising a sequence that shares at least 80% homology with at least one of SEQ ID NO:3 and SEQ ID NO:4 or a functional equivalent thereof.

19. The method of claim 12, wherein immune response is generated in the pig against one or more influenza strains selected from the group consisting of H1N1, H1N2, and H3N2.

20. The method of claim 12, wherein said second swine influenza vaccine comprises an inactivated swine influenza vaccine.

21. The method of claim 20, wherein said second vaccine is selected from the group consisting of a whole inactivated virus vaccine, a split virus vaccine, a subunit vaccine, a live attenuated vaccine, and a virosome vaccine.

22. The method of claim 12, wherein said second vaccine comprises the same DNA vaccine.

23-28. (canceled)
